(19) United States
(12) Patent Application Publication
(10) Pub. No.: US 2026/0191438 A1
(43) Pub. Date: Jul. 9, 2026

(54) METHOD FOR DETERMINING A SUBJECT'S ANALYTE LEVEL

(71) Applicants: Roche Diabetes Care, Inc., Indianapolis, IN (US); Roche Diabetes Care GmbH, Mannheim (DE)

(72) Inventors: Jennifer Fisher, Fishers, IN (US); Thomas Miyakawa, Indianapolis, IN (US); Jeffrey M. Bender, Fishers, IN (US); Siva Chittajallu, Indianapolis, IN (US); Herbert Fink, Mannheim (DE)

(21) Appl. No.: 19/559,094

(22) Filed: Mar. 6, 2026

Related U.S. Application Data
(63) Continuation of application No. PCT/EP2024/075166, filed on Sep. 10, 2024.
(60) Provisional application No. 63/582,305, filed on Sep. 13, 2023.

(30) Foreign Application Priority Data
Oct. 2, 2023 (EP) 23201258.3

(51) Int. Cl. A61B 5/145; A61B 5/00; A61B 5/08; A61B 5/097

(52) U.S. Cl. CPC A61B 5/14532; A61B 5/0024; A61B 5/082; A61B 5/097

(57) ABSTRACT

A method for determining a subject's analyte level is disclosed. The method includes the steps of: a) non-invasively detecting a first amount of at least one first volatile organic marker originating from a first source of the subject; b) non-invasively detecting a second amount of at least the first volatile organic marker originating from a second source of the subject, wherein the second source is different from the first source; and c) determining the subject's analyte level based on the first amount and on the second amount.

### CROSS REFERENCE TO RELATED APPLICATIONS

This is a continuation of application serial no. PCT/EP2024/075166 filed Sep. 10, 2024 which claims priority to EP 23 201 258.3 filed Oct. 2, 2023, and U.S. Ser. No. 63/582,305 filed Sep. 13, 2023, the disclosures of all of which are hereby incorporated herein by reference.

### BACKGROUND

### Technical Field

This disclosure relates generally to a non-invasive determination of a subject's blood glucose level based on detecting volatile organic markers in exhaled breath or other gaseous emanation from the subject.

### Background Art

The monitoring of blood glucose levels is crucial for diabetes management. A typical glucose measurement method involves piercing the skin, typically a finger, to draw blood and applying the blood to a chemically active disposable medium. To avoid the necessity of repeatedly piercing the skin, different non-invasive blood glucose monitoring technologies have also been proposed.

The prior art describes the use of measuring volatile organic compounds (VOC) in different settings, thus originating from different sources, e.g., breath samples, and being detected via different methods, for example electrochemically. Furthermore, it is known to combine the detection of different VOCs, e.g., by measuring different VOCs simultaneously via one or more detection devices.

U.S. Pat. No. 10,526,633B2 describes plant/plant pathogen volatile compound electrochemical sensors, plant/plant pathogen volatile detection systems, and methods for detecting stress-induced plant volatile compounds and/or a plant-pathogen emitted volatile compounds.

U.S. Pat. No. 9,551,712B2 describes sets of VOCs for breath analysis. Methods of use thereof in diagnosing, monitoring or prognosing breast cancer, head and neck cancer, prostate cancer or colon cancer are disclosed.

US20210341461A1 describes a method for the early detection and monitoring of the progression of cancer by detecting breath biomarkers. The method comprises assessing the activity of an aldo-keto reductase by measuring the concentration of an exogenous substrate for said enzyme and/or measuring the concentration of a metabolite of said substrate in exhaled breath of a subject. Preferably, the cancer is lung cancer.

EP2270496A1 describes an identification of markers for the disease conditions related to cystic fibrosis (CF). The uses of such markers in diagnosis and a novel method for their identification are described. It was discriminated between patients with cystic fibrosis and normal individuals by determining whether the exhaled air contained markers selected from that set. Is was found that these markers can be used in the early diagnosis of cystic fibrosis.

WO2022101598A1 describes an apparatus for detecting the presence in an enclosed environment of a subject or subjects infected with viral, bacterial and/or parasitic disease or diseases, the apparatus comprising: (a) an air sampling unit able to take an air sample of the atmosphere in the enclosed environment and to divert said sample for sensing; (b) a selected definitive sensor set comprising at least two sensors reactive to the presence of specific odours or Volatile Organic Compounds (VOCs) in the air sample taken from the environment; (c) a processing unit comprising a pattern recognition analyser, wherein the pattern recognition analyser receives output signals of the sensor set, compares them to disease-specific patterns derived from a database of response patterns of the sensor set exposed to the totality of the bodily emissions of subjects with known disease or diseases, wherein each of the disease-specific patterns is characteristic of a particular disease, selected from bacteriological, viral and parasitic disease, and selects a closest match between the output signals of the sensor set and the disease-specific pattern; and (d) a control system that triggers the sampling of the air space of the environment at pre-determined times or intervals for rendering the apparatus entirely automatic and self-contained in operation.

WO2012040318A2 describes compositions, methods and kits for detection of melanoma and determination of melanoma margins. It is related to a panel of volatile metabolic biomarkers that can be used in the diagnosis of melanoma skin cancer. An approach to detect melanoma is based on volatile by-products of altered cancer metabolism. Further provided is a method for identifying molecules useful in the detection of melanoma and sets a foundation for development of a non-invasive detection technology, a biosensor, for melanoma diagnosis.

CN115297768A describes a method of determining whether a subject has mild cognitive impairment (MCI) or Alzheimer's disease, the method comprising measuring the concentration of one or more VOCs in an exhaled sample of the subject and comparing the concentration to a reference concentration.

EP4085832A1 describes a method to determine a subject's blood glucose level non-invasively by analyzing an exhaled breath or another gaseous emanation from the subject, comprising non-invasively detecting an amount of at least one volatile organic marker in the subject's exhaled breath or the subject's other gaseous emanation as marker data and determining the subject's blood glucose level based on the marker data, wherein the volatile organic marker is selected from a group of markers, such as indole, for which the amount of the volatile organic marker is negatively correlated to the blood glucose level.

Despite of the advantages and the progress achieved by the above-mentioned developments, some significant technical challenges remain regarding an accuracy of the analyte determination.

### SUMMARY

It is therefore desirable to provide a method for determining a subject's analyte level and a system for determining a subject's analyte level which at least partially address the above-mentioned technical challenges. Specifically, a method for determining a subject's analyte level and a system for determining a subject's analyte level are desirable which increase an accuracy of the determination of the subject's analyte level.

This problem is addressed by a method for determining a subject's analyte level and a system for determining a subject's analyte level with features of the present disclosure. Advantageous embodiments which might be realized in an isolated fashion or in any arbitrary combinations are described throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, a method for determining a subject's analyte level is disclosed.

The term “subject” as used herein is a broad term and is to be given its ordinary and customary meaning to a person of ordinary skill in the art and is not to be limited to a special or customized meaning. The term exemplarily relates to a person intending to monitor an analyte value, such as a glucose value. In an embodiment, the term specifically may refer, without limitation, to a person applying the method. For example, the subject may be a patient suffering from a disease such as diabetes. The subject may also be referred to as user or as patient. However, in an embodiment, the person applying the method is different from the subject.

The term “analyte” as used herein is a broad term and is to be given its ordinary and customary meaning to a person of ordinary skill in the art and is not to be limited to a special or customized meaning. The term specifically may refer, without limitation, to an arbitrary element, component or compound which may be present in a body of the subject and a presence and/or a concentration of which may be of interest for the subject or medical staff such as a medical doctor. Particularly, the analyte may be or may comprise an arbitrary chemical substance or chemical compound which may take part in a metabolism of the subject, such as at least one metabolite. Specifically, the analyte may be glucose, more specifically blood glucose. Additionally or alternatively, however, other types of analytes may be used and/or any combination of analytes may be determined.

The terms “determining an analyte level” and “detecting an analyte” as used herein are broad terms and are to be given its ordinary and customary meaning to a person of ordinary skill in the art and are not to be limited to a special or customized meaning. The terms specifically may refer, without limitation, to a process of determining a presence and/or a quantity and/or a concentration of at least one analyte. Thus, the detection may be or may comprise a qualitative detection, simply determining the presence of the at least one analyte or the absence of the at least one analyte, and/or may be or may comprise a quantitative detection, which determines the quantity and/or the concentration of the at least one analyte. As a result of the detection, at least one signal may be produced which characterizes an outcome of the detection.

The term “concentration” may generally refer to an amount of an arbitrary substance in another medium. Specifically, the term “concentration” may refer to an amount of a dissolved or non-dissolved substance in a fluidic medium. The concentration may be described qualitatively, exemplarily as a mass concentration, as a molar concentration, as a number concentration or as a volume concentration. Thereby, the concentration may be quantified as a mass of the substance divided by the volume of the other medium, as an amount of the substance in moles divided by the volume of the other medium, as a number of the entities of the substance divided by the volume of the other medium and as a volume of the substance divided by the volume of the other medium, respectively. However, other kinds of descriptions may be feasible, such as by using a normality, a molality, a mole fraction, a mole ratio, a mass fraction, or a mass ratio. Thereby, the concentration may be quantified as a molar concentration divided by an equivalence factor, as an amount of the substance divided by a mass of a solvent of the other medium, as an amount of the substance divided by the mass of the other medium, as an amount of the substance divided by a total amount of all components of the other medium, as an amount of the substance divided by a total amount of all other components of the other medium, as a mass of the substance divided by a mass of all components of the other medium and as a mass of the substance divided by a mass of all other components of the other medium, respectively.

The concentration of glucose in the blood of the subject may be dependent on events which increase or decrease the concentration of glucose such as an intake of food or physical activity. Thus, the concentration of glucose in the blood may be describable as time-dependent concentration. Generally, the term “time-dependent concentration” may refer to a property of a concentration of varying or changing over time. Thus, when evaluating the concentration at a first point in time, the concentration may have a first value and when evaluating the concentration at a second point in time, the concentration may have a second value which may be differ from the first value. The second value may be higher or lower than the first value.

The method comprises the method steps as listed as follows. The method steps may be performed in the given order. However, other orders of the method steps are feasible. Further, one or more of the method steps may be performed in parallel and/or on a timely overlapping fashion. Further, one or more of the method steps may be performed repeatedly. Further, additional method steps may be present which are not listed.

The method comprises the steps of:
a) non-invasively detecting a first amount of at least one first volatile organic marker originating from a first source of the subject;
b) non-invasively detecting a second amount of at least the first volatile organic marker originating from a second source of the subject, wherein the second source is different from the first source; and
c) determining the subject's analyte level based on the first amount and on the second amount.

The term “non-invasively detecting” as used herein is a broad term and is to be given its ordinary and customary meaning to a person of ordinary skill in the art and is not to be limited to a special or customized meaning. The term specifically may refer, without limitation, to a process of determining a presence and/or a quantity and/or a concentration of at least one analyte without inserting a component or a part of the component of a detection unit such as an analyte sensor into a body, specifically into a body tissue, of the subject. Thus, the detection unit including the analyte sensor may fully stay outside, specifically fully outside, of the body, specifically of the body tissue. The detection unit may exemplarily be in close contact with a skin of the subject. Exemplarily, the detection unit may be attached to a subject's skin via an adhesive or may be attached to the subject's skin via a wristband. Further, the detection unit may be placeable close to nostrils and/or to a mouth of the subject. Further details on the embodiment of the detection unit are given below in more detail.

The terms “first amount” and “second amount” may be considered as nomenclature only, without numbering or ranking the named elements, without specifying an order and without excluding a possibility that several kinds of first amounts and second amounts may be present. Further, additional amounts such as one or more third amounts may be present. Further details on additional amounts are given below in more detail.

The term “amount” as used herein is a broad term and is to be given its ordinary and customary meaning to a person of ordinary skill in the art and is not to be limited to a special or customized meaning. The term specifically may refer, without limitation, to a quantity and/or a concentration of a substance. As a result of a determination of the amount, at least one signal may be produced which characterizes an outcome of the detection, such as at least one measurement signal. It is understood that the detected amount of each volatile organic marker comprises a single value or a multitude of values over time wherein each value represents a single measurement or an average over two or more measurements. It is also understood that the amount of the volatile organic marker is either determined in absolute values or in relative values, e.g., determining an amount of change of a marker concentration.

The term “first volatile organic marker” may be considered as nomenclature only, without numbering or ranking the named element, without specifying an order and without excluding a possibility that several kinds of first volatile organic markers may be present. Further, additional volatile organic markers such as one or more second volatile organic markers may be present. Further details on additional volatile organic markers are given below in more detail.

The term “volatile organic marker”, also called volatile organic compound (VOC), as used herein is a broad term and is to be given its ordinary and customary meaning to a person of ordinary skill in the art and is not to be limited to a special or customized meaning. The term specifically may refer, without limitation, to an organic chemical that has a high vapor pressure or rather a low boiling point. Thus, VOCs are volatile at rather low temperatures such as room temperature for example. The human body is a major source of VOCs that originate from different parts and processes within the body. So-called endogenous VOCs originate from metabolic processes within the body. An important organ involved in generating and transforming such endogenous VOCs is the liver. VOCs originating from the environment are called exogenous VOCs, e.g., entering the body via respiratory air, food intake or diffusion through skin or originating from the microbiome, e.g., of the digestive tract.

VOCs generated or absorbed in various parts of the body enter the bloodstream and pass into the exhaled breath by gas exchange in the lungs. VOCs from skin originate from secretion of eccrine, sebaceous or apocrine glands.

It is noted, that a VOC may as well represent a derivative of a compound of interest, i.e., a compound originating from an endogenous process, or a fragment of a compound of interest, said fragment being formed by cleavage of the molecule during the analytical process.

Specifically, the first volatile organic marker may be selected from a group of markers for which the amount of the volatile organic marker is negatively correlated to the glucose level. Surprisingly, a negative correlation between the volatile organic marker amount and the blood glucose level has proven to be particularly beneficial for the reliability, responsiveness and sensitivity of the method.

The negative correlation may only be achieved by compounds that do have a functional role in the insulin response. The amount of such compounds, i.e., markers, in breath or other emanation are essentially independent of external influences or individual characteristics of the patient.

Specifically, the first volatile organic marker may be one of indole (C₈H₇N), a partly saturated derivative of indole, fully saturated derivative of indole and a true fraction of indole.

Indole is an aromatic heterocyclic organic compound with formula C₈H₇N. It is widely distributed in the natural environment and can be produced by a variety of bacteria, such as E. coli, which are usually present in the human intestine. Furthermore, indole is the most abundant metabolite produced from digestion of tryptophan. As many bacteria present in the human intestine can synthesize indole from tryptophan due to the enzyme tryptophanase the indole level in the human intestine is constant.

Moreover, indole is a possible signaling molecule for stimulation of Glucagon-Like-Protein-1 (GLP-1), which is needed for an accurate insulin response. In the presence of glucose, indole diffuses into intestinal cells and increases GLP-1 secretion by blocking K⁺ channels. This results in a decrease of indole concentration in the extracellular space, a decrease that is then observed in breath. As soon as extracellular glucose levels drop, indole diffuses out of cells again and detaches from K⁺ channels. These reversible mechanisms indicate that indole acts as a signal molecule only and is not metabolized. A result is the inverse progression of indole compared to blood glucose levels, i.e., the negative correlation. Furthermore, said correlation enables a monitoring of the blood glucose levels in real-time as any change in the blood glucose concentration occurs simultaneously to a change of the amount of Indole. “Simultaneously” here means that the times at which a change in the two concentrations occurs are separated by a maximum of five minutes.

A derivative is a compound that is derived from a similar compound by a chemical reaction. Some derivatives of indole, e.g., aliphatic C₈-amines like cyclohexyl-ethylamine (C₈H₁₉N) or octylamine (C₈H₁₇N) or isomers thereof show a corresponding correlation to blood glucose as well as the described advantage. This also holds for some fragments, e.g., benzenes, wherein fragments denominate products of fragmentation, i.e., the dissociation of energetically unstable molecular ions formed from passing the molecules in the ionization chamber of a mass spectrometer. The term “true fragment” is used to indicate that said fragment originates from the dissociation of the claimed compound, i.e., indole.

Indole has a molecular weight of ~117.1 g/mol. Thus, when detecting Indole via PTR-ToF-MS the mass-to-charge ratio after proton transfer for PTR-ToF-MS is m/z=118.1 due to the additional H⁺.

Derivatives are for example cyclohexyl-ethylamine C₈H₁₇N with a mass-to-charge ratio of m/z=128.14 after protonation with H⁺ or octylamine C₈H₁₉N with a mass-to-charge ratio of m/z=130.15 after protonation.

Fragments of indole are for example benzene C₆H₆ with a mass-to-charge ratio of m/z=79.055 after protonation with H⁺ and C₇H₈ with a mass-to-charge ratio of m/z=93.069 after protonation.

Further, specifically, the first volatile organic marker may be selected from the group consisting of: formaldehyde, methanol. Further, the first volatile organic marker may be selected from the group consisting of: acrolein, acetic acid, butanone, propionic acid, phenol, acetone, propanamide, butyric acid. Also other volatile organic markers may be possible.

The term “source” as used herein is a broad term and is to be given its ordinary and customary meaning to a person of ordinary skill in the art and is not to be limited to a special or customized meaning. The term specifically may refer, without limitation, to an area of or in close proximity to a subject's body where an arbitrary substance comes from or is present. Further, the term “source” may refer to an excretion or a secretion of the substance from the subject's body, specifically from a part or an area of the subject's body and/or from an organ of the subject's body such as a skin, a nose and/or a mouth. Specifically, the term “source” refer to a part or an area of or in close proximity to the subject's body and/or to an excretion or a secretion of the substance from the subject's body, specifically from a part or an area of the subject's body and/or from an organ of the subject's body such as a skin, a nose and/or a mouth where the at least one first volatile organic marker and/or one or more other volatile organic markers come from or are present.

The terms “first source” and “second source” may be considered as nomenclature only, without numbering or ranking the named elements, without specifying an order and without excluding a possibility that several kinds of first sources and second sources may be present. Further, additional sources such as one or more third sources may be present. As outlined above, the second source is different from the first source. Thus, the first source and the second source may be different kinds of sources. The first source and the second source may be different areas of or in close proximity to a subject's body where the first volatile organic marker comes from or is present. Specifically, the first source and the second source may refer to different organs of the subject where the first volatile organic marker comes from or is present. Specifically, the first source may be exhaled breath, specifically exhaled breath originating from a subject's nostrils and/or from a subject's mouth and the second source may be another source of gaseous emanation which is different from exhaled breath, or vice versa. Thus, the second source may be exhaled breath, specifically exhaled breath originating from the subject's nostrils and/or from the subject's mouth and the first source may be the other source of gaseous emanation which is different from exhaled breath. Specifically, the other source of gaseous emanation which is different from exhaled breath may correspond to a secretion from a skin, specifically from a skin area, of the subject's body. Exemplarily, the skin, specifically the skin area, may be selected from the group consisting of: head, chest, back, armpit, waist, arm or genital area. However, also other skin areas may be possible.

As outlined above, in step c), the subject's analyte level may be determined based on the first amount and on the second amount. The subject's analyte level may be determined from the detected amount of the first volatile organic marker in the exhaled breath and the other emanation due to a correlation between said entities. It is understood that the correlation provides for values of the analyte such as blood glucose derived from absolute or relative values of the first volatile organic marker concentration in the exhaled breath and the other emanation. Exemplarily, first volatile organic data may be normalized based on a reference signal, e.g., derived from ambient air or from another marker in the same sample of exhaled breath or from another sample of exhaled breath or other emanation. Exemplarily, a set-up to perform the method is taught and/or trained based on individual metabolic processes, e.g., creating individual profiles for the subject. For example, the training involves selecting the volatile organic marker from a group of volatile organic markers also comprising derivatives or fragments of endogenous compounds.

The method may further comprise:
d) non-invasively detecting a third amount of at least a second volatile organic marker originating from the first source of the subject; and
e) non-invasively detecting a fourth amount of the at least one second volatile organic marker originating from the second source of the subject.

Specifically, steps d) and e) may be performed before conducting step c). In step c), the subject's analyte level may be detected based on the first amount, the second amount, the third amount and the fourth amount. Specifically, the second volatile organic marker may be different from the first volatile organic marker. Thus, the first volatile organic marker and the second volatile organic marker may be different kinds of volatile organic markers. Specifically, the first volatile organic marker may be indole and the second volatile organic marker may be formaldehyde or methanol. However, also other embodiments may be possible. Specifically, the analyte may be glucose and the method may further comprise detecting a state of hyperglycemia or hypoglycemia based on first amount, the second amount, the third amount and the fourth amount.

Specifically, the first volatile organic marker and the second volatile organic marker may be formaldehyde and methanol. Further, the first volatile organic marker and the second volatile organic marker may be formaldehyde and acrolein. Further, the first volatile organic marker and the second volatile organic marker may be formaldehyde and propanamide. Further, the first volatile organic marker and the second volatile organic marker may be acetone and indole. With all these sets, a mean absolute relative difference (MARD) of 21% for diabetes type 1 and a MARD of 13% for diabetes type 2 could be achieved.

An assessment of a measurement accuracy of blood glucose meters and continuous glucose systems is commonly primarily carried out via the mean absolute relative difference (MARD). A value determined with a measuring system is commonly compared with a measured value of a reference device, preferably a product that can be considered a standard because of its high accuracy. A difference between the two values gives a relative deviation. An amount of the difference is commonly taken, because this can assume both positive and negative values. The MARD may be regarded as a mean value over each relative deviation.

The method may further comprise:
f) non-invasively detecting a fifth amount of at least a third volatile organic marker originating from the first source of the subject; and
g) non-invasively detecting a sixth amount of the at least one third volatile organic marker originating from the second source of the subject.

Specifically, steps f) and g) may be performed before conducting step c). In step c), the subject's analyte level may be detected based on the first amount the second amount, the third amount, the fourth amount, the fifth amount and the sixth amount. Specifically, the third volatile organic marker may be different from the first volatile organic marker and from the second volatile organic marker. Thus, the first volatile organic marker, the second volatile organic marker and the third volatile organic marker may be different kinds of volatile organic markers. Specifically, the first volatile organic marker may be indole, the second volatile organic marker may be formaldehyde and the third volatile organic marker may be methanol or vice versa. However, also other embodiments may be possible. Specifically, the analyte may be glucose and the method may further comprise detecting a state of hyperglycemia or hypoglycemia based on first amount, the second amount, the third amount, the fourth amount, the fifth amount and the sixth amount.

Specifically, the first volatile organic marker, the second volatile organic marker and the third volatile organic marker may be formaldehyde, indole and methanol. Further, the first volatile organic marker, the second volatile organic marker and the third volatile organic marker may be formaldehyde, indole and acrolein. Further, the first volatile organic marker, the second volatile organic marker and the third volatile organic marker may be formaldehyde, indole and acetic acid. Further, the first volatile organic marker, the second volatile organic marker and the third volatile organic marker may be formaldehyde, indole and propanamide. Further, the first volatile organic marker, the second volatile organic marker and the third volatile organic marker may be formaldehyde, indole and propionic acid. Further, the first volatile organic marker, the second volatile organic marker and the third volatile organic marker may be formaldehyde, indole and butyric acid. Further, the first volatile organic marker, the second volatile organic marker and the third volatile organic marker may be methanol, indole and actone. With all these sets, a mean absolute relative difference (MARD) of 17% for diabetes type 1 and a MARD of 10% for diabetes type 2 could be achieved.

The method may further comprise:
h) non-invasively detecting a seventh amount of at least a fourth volatile organic marker originating from the first source of the subject; and
i) non-invasively detecting an eighth amount of the at least one fourth volatile organic marker originating from the second source of the subject.

Specifically, steps h) and i) may be performed before conducting step c). In step c), the subject's analyte level may be detected based on the first amount, the second amount, the third amount, the fourth amount, the fifth amount, the sixth amount, the seventh amount and the eighth amount. Specifically, the fourth volatile organic marker may be different from the first volatile organic marker, from the second volatile organic marker and from the third volatile organic marker. Thus, the first volatile organic marker, the second volatile organic marker, the third volatile organic marker and the fourth organic marker may be different kinds of volatile organic markers. Specifically, the analyte may be glucose and the method may further comprise detecting a state of hyperglycemia or hypoglycemia based on first amount, the second amount, the third amount, the fourth amount, the fifth amount, the sixth amount, the seventh amount and the eighth amount.

Specifically, the first volatile organic marker, the second volatile organic marker, the third volatile organic marker and the fourth volatile organic marker may be formaldehyde, methanol, indole and propanamide. Further, the first volatile organic marker, the second volatile organic marker, the third volatile organic marker and the fourth volatile organic marker may be formaldehyde, methanol, indole and propionic acid. Further, the first volatile organic marker, the second volatile organic marker, the third volatile organic marker and the fourth volatile organic marker may be formaldehyde, methanol, indole and acetic acid. Further, the first volatile organic marker, the second volatile organic marker, the third volatile organic marker and the fourth volatile organic marker may be formaldehyde, methanol, indole and butyric acid. Further, the first volatile organic marker, the second volatile organic marker, the third volatile organic marker and the fourth volatile organic marker may be formaldehyde, methanol, indole and acrolein. Further, the first volatile organic marker, the second volatile organic marker, the third volatile organic marker and the fourth volatile organic marker may be formaldehyde, indole, butyric acid and propanamide. Further, the first volatile organic marker, the second volatile organic marker, the third volatile organic marker and the fourth volatile organic marker may be formaldehyde, indole, butyric acid and propionic acid. With all these sets, a mean absolute relative difference (MARD) of 14% for diabetes type 1 and a MARD of 8% for diabetes type 2 could be achieved.

The method may further comprise:
j) non-invasively detecting a ninth amount of at least a fifth volatile organic marker originating from the first source of the subject; and
k) non-invasively detecting a tenth amount of the at least one fifth volatile organic marker originating from the second source of the subject.

Specifically, steps j) and k) may be performed before conducting step c). In step c), the subject's analyte level may be detected based on the first amount, the second amount, the third amount, the fourth amount, the fifth amount, the sixth amount, the seventh amount, the eighth amount, the ninth amount and the tenth amount. Specifically, the fifth volatile organic marker may be different from the first volatile organic marker, from the second volatile organic marker, from the third volatile organic marker and from the fourth volatile organic marker. Thus, the first volatile organic marker, the second volatile organic marker, the third volatile organic marker, the fourth volatile organic marker and the fifth volatile organic marker may be different kinds of volatile organic markers. Specifically, the analyte may be glucose and the method may further comprise detecting a state of hyperglycemia or hypoglycemia based on first amount, the second amount, the third amount, the fourth amount, the fifth amount, the sixth amount, the seventh amount, the eighth amount, the ninth amount and the tenth amount

Specifically, the first volatile organic marker, the second volatile organic marker, the third volatile organic marker, the fourth volatile organic marker and the fifth volatile organic marker may be formaldehyde, methanol, indole, propanamide and butyric acid. Further, the first volatile organic marker, the second volatile organic marker, the third volatile organic marker, the fourth volatile organic marker and the fifth volatile organic marker may be formaldehyde, methanol, indole, propanamide and phenol. Further, the first volatile organic marker, the second volatile organic marker, the third volatile organic marker, the fourth volatile organic marker and the fifth volatile organic marker may be formaldehyde, methanol, indole, propionic acid and phenol. Further, the first volatile organic marker, the second volatile organic marker, the third volatile organic marker, the fourth volatile organic marker and the fifth volatile organic marker may be methanol, acetone, propanamide, butyric acid and indole. Further, the first volatile organic marker, the second volatile organic marker, the third volatile organic marker, the fourth volatile organic marker and the fifth volatile organic marker may be formaldehyde, methanol, indole, acrolein and propanamide. Further, the first volatile organic marker, the second volatile organic marker, the third volatile organic marker, the fourth volatile organic marker and the fifth volatile organic marker may be formaldehyde, methanol, indole, acetone and propanamide. Further, the first volatile organic marker, the second volatile organic marker, the third volatile organic marker, the fourth volatile organic marker and the fifth volatile organic marker may be formaldehyde, methanol, indole, butanone and propanamide. Further, the first volatile organic marker, the second volatile organic marker, the third volatile organic marker, the fourth volatile organic marker and the fifth volatile organic marker may be formaldehyde, methanol, indole, acetic acid and propionic acid. Further, the first volatile organic marker, the second volatile organic marker, the third volatile organic marker, the fourth volatile organic marker and the fifth volatile organic marker may be formaldehyde, methanol, indole, propionic acid and butyric acid. Further, the first volatile organic marker, the second volatile organic marker, the third volatile organic marker, the fourth volatile organic marker and the fifth volatile organic marker may be formaldehyde, methanol, indole, propanamide and propionic acid. Further, the first volatile organic marker, the second volatile organic marker, the third volatile organic marker, the fourth volatile organic marker and the fifth volatile organic marker may be formaldehyde, methanol, indole, acetone and phenol. With all these sets, a mean absolute relative difference (MARD) of 12% for diabetes type 1 and a MARD of 6-7% for diabetes type 2 could be achieved.

Specifically, in step a) the first amount of the at least one first volatile organic marker originating from the first source of the subject may be detected continuously or discontinuously. Further, specifically, in step b) the second amount of the at least one first volatile organic marker originating from the second source of the subject may be detected continuously or discontinuously. Specifically, in step a) the first amount of the at least one first volatile organic marker originating from the first source of the subject may be detected discontinuously and in step b) the second amount of the at least one first volatile organic marker originating from the second source of the subject may be detected continuously. Specifically, the first source may be exhaled breath and in step a) the first amount of the at least one first volatile organic marker originating from the first source of the subject may be detected discontinuously. Further, specifically, the second source may be another source of gaseous emanation which is different from exhaled breath such as the subject's skin area and in step b) the second amount of the at least one first volatile organic marker originating from the second source of the subject may be detected continuously. When the first amount and/or the second amount may be detected continuously, a data stream may be acquired. The term “data stream” may specifically refer to a sequence or to an assembly of data elements, specifically such as signals, more specifically such as electrical signals, which are available or provided to an arbitrary device over time or in a time-dependent manner. The data stream may be a continuous data stream. However, the data stream may comprise or may have one or more gaps wherein, during the gaps, no data may be transferred to the device. Further, a number of data elements or signals per time unit which are transferred to the device may vary over time. When the first amount and/or the second amount is detected discontinuously, only a single data element is acquired or several data elements are acquired during a predefined time period.

The method may further comprise displaying the subject's analyte level to the subject such as via at least one displaying device such as a smartwatch or a smartphone. Thus, the subject may be informed about his or her glycemic condition such as in the morning. For details are given below.

The method may further comprise comparing the subject's analyte level to a predefined threshold value or to a predefined tolerance interval being defined by a first threshold value and a second threshold value being different from the first threshold value. The term “threshold value” may generally refer to a defined, a predefined or a determinable numeral value which is used as a comparison to be compared with one or more items of information such as with data and/or with measurement values in order to derive at least one secondary item of information. As an example, the threshold value may define a comparison value which is compared with measurement data and/or other items of information, wherein, depending on the comparison, one or more results may be stated. As an example, the threshold value may define a comparison value, wherein, once the at least one item of information reaches the comparison value, exceeds the comparison value or falls below the comparison value, one or more results are stated. The terms “first threshold value” and “second threshold value” may be considered as nomenclature only, without numbering or ranking the named elements, without specifying an order and without excluding a possibility that several kinds of first threshold values and second threshold values may be present. Further, additional threshold values such as one or more third threshold values may be present. A target range may be defined by the first threshold value and by the second threshold value. The target range may define a desired range, e.g., the tolerance interval, for the analyte level. Thereby, the subject may be in optimal healthy conditions with regard to the desired analyte level. The term “predefined” may specifically refer to the circumstance that the threshold value or the tolerance interval is defined, e.g., predetermined and known, prior to application of the method.

A warning signal may be output in case the subject's analyte level is outside the predefined tolerance interval. The warning signal may provide indications about actions required by the subject such as medication intake or food intake. As further used herein, the term “signal” may refer to an arbitrary indication which is transferable from one element to another element, specifically in order to indicate, warn, direct or to command. Thus, the indication may comprise at least one item of information. Specifically, the signal may be or may comprise at least one of an electronic signal, a visual signal, an acoustic signal or a vibrational signal.

The method may comprise acquiring a continuous series of consecutive measurements. The measurements may be used for predicting the subject's analyte level in the future such as within a few minutes. The method may comprise predicting the subject's analyte level by using at least one mathematical model. When the analyte is glucose, specifically blood glucose, there may be a delay in time between a change of the glucose level in the blood and a change of the volatile organic marker originating from breath. Typically, the change of the volatile organic marker originating from breath may take place sooner than the change of the glucose level in the blood. The delay in time may exemplarily be about 5 minutes. The delay in time may be considered by the mathematical model.

Thus, the method according to the present disclosure may be advantageous in comparison to a detection of the subject's analyte level from blood or interstitial fluid. During events known to have an influence on the subject's analyte level such as a meal intake, the method according to the present disclosure may have a time advantage in comparison to the detection of the subject's analyte level from blood or interstitial fluid.

The mathematical model may comprise at least one machine learning architecture such as including neuronal networks, specifically to improve an accuracy of glucose readings. The mathematical model may specifically comprise at least one linear and/or non-linear time-series model, specifically to improve a prediction performance. The method may specifically use recurrent neural networks (RNN), more specifically long short-term memory (LSTM) networks. Further, the mathematical model may comprise at least one double-logarithmical function.

The method may further comprise a field calibration. The field calibration may include a procedure to establish a personalized algorithm for a specific subject. The field calibration may include a calibration with an individual subject. The individual subject may undergo some tests such as described below in more detail. Further, individual subject characteristics may be considered such as described below in more detail. A personalized mathematical model for the subject may be established and/or the mathematical model may be chosen from a number of readily predetermined mathematical models. Specifically, the field calibration may comprise classifying and/or clustering of the subject to a specific mathematical model, specifically chosen from the number of the readily predetermined mathematical models. Specifically, subjects may be clustered in individual clusters such as in about 5 individual clusters and may be clustered in individual base models developed for each cluster. The models may be augmented by additional features obtained from first data points from the individual subject. For field calibration, single breath measurements may be made by the subject. Further, additionally or alternatively, for field calibration, an entire series of breath measurements during an event with analyte variation may be used. Specifically, the analyte may be glucose and the event with analyte variation may be an oral glucose tolerance test (OGTT). Further, for field calibration, single analyte measurements or an entire series of measurements during an event with analyte variation may be made by the subject via another method such as a determining of the subject's analyte level via analyte detection in a body fluid such as blood or interstitial fluid. Further, additionally or alternatively, for field calibration, further subject characteristics may be considered. The subject characteristics may be selected from the group consisting of: age, gender, body mass index (BMI), weight, medication, type of diabetes, smoking behavior. Also other and/or further subject characteristics may be possible. The field calibration may comprise providing feedback to the subject about a goodness of a fit to a specific mathematical model. In case the goodness is not sufficient, the field calibration may comprise providing options to the subject for improving the field calibration such a consideration of further and/or different subject characteristics and/or measurements. The providing of options may also include a recommendation to use a different device with a different method for determining the subject's analyte level.

The method may comprise using a data base. The data base may comprise a plurality of different mathematical models. Specifically, the mathematical models may be respectively trained for a special group of patients. Specifically, the mathematical models may be respectively trained on group-specific training data. The method may comprise selecting a mathematical model from the data base, specifically based on the subject's parameters, more specifically based on the field calibration as outlined above. Specifically, the method may comprise an entering of parameters by the subject via a user interface.

The method may further comprise detecting an event known to have an effect on the subject's analyte level. Specifically, the method may comprise detecting an event known to increase the subject's analyte level. Specifically, the analyte may be glucose and the method may comprise detecting a meal intake by the subject. The meal intake may also be detected in case the meal intake is not recorded by the subject. The method may further comprise estimating a meal size and/or a glycemic index of the meal. The detecting of the event known to have an effect on the subject's analyte level may specifically be based on characteristics in an analyte trend. Specifically, the detecting of the meal intake may be based on a characteristic rise of glucose. Further, the method may comprise considering contextual information such as events known to have an effect on the subject's analyte level, exemplarily a meal intake or a subject's physical activity, for prediction of the analyte trend. As described above, the contextual information may be detected by the method. However, additionally or alternatively, the contextual information may be entered by the subject via the user interface. Further, the method may comprise recognizing time phases with a reduced prediction accuracy of glucose levels such as time phases with fast analyte changes and the method may comprise providing information about the time phases to the subject.

The analyte may be glucose and the method may further comprise giving an estimate for an insulin dosing, specifically for an improved insulin dosing. The estimate for the insulin dosing may be based on average values of the glucose level, specifically during specific time intervals such as during sleep times or specific time intervals of the day. Further, the estimate for the insulin dosing may be based on the rise of glucose after a meal intake. Also other options may be possible.

The method may further comprise at least one failsafe step. The term “failsafe step” as used herein is a broad term and is to be given its ordinary and customary meaning to a person of ordinary skill in the art and is not to be limited to a special or customized meaning. The term may, specifically, refer, without limitation, to at least one step ensuring to prevent generating and/or determining and/or displaying unreliable or even false measurement values. During the failsafe step a recognition of an unrealistic measurement value may occur.

In a further aspect of the present disclosure, a system for determining a subject's analyte level is disclosed. The system is configured for executing the method according as described above or as will further be described below in more detail.

The term “system” as used herein is a broad term and is to be given its ordinary and customary meaning to a person of ordinary skill in the art and is not to be limited to a special or customized meaning. The term specifically may refer, without limitation, to a group of at least two elements or components which are capable of interacting in order to perform at least one common function, in the present case in order to perform the determination of the subject's analyte level or to contribute to the determination of the subject's analyte level. The system specifically may comprise an assembly of two or more components capable of interacting with each other, such as in order to perform one or more diagnostic purposes, such as in order to perform a medical analysis. The system generally may also be referred to as an assembly or as a kit.

The system comprises:
a first volatile organic marker detection unit configured for non-invasively detecting the first amount of the at least one first volatile organic marker originating from the first source of the subject;
a second volatile organic marker detection unit configured for non-invasively detecting the second amount of the at least one first volatile organic marker originating from the second source of the subject; and
at least one evaluation device configured for determining the subject's analyte level based on the first amount and on the second amount.

The terms “first volatile organic marker detection unit” and “second volatile organic marker detection unit” may be considered as nomenclature only, without numbering or ranking the named elements, without specifying an order and without excluding a possibility that several kinds of first or second volatile organic marker detection units may be present. Further, additional volatile organic marker detection units such as one or more third volatile organic marker detection units may be present. The term “volatile organic marker detection unit” as used herein is a broad term and is to be given its ordinary and customary meaning to a person of ordinary skill in the art and is not to be limited to a special or customized meaning. The term specifically may refer, without limitation, to an arbitrary element which is adapted to perform a process of detection and/or which is adapted to be used in a process of detection. Thus, volatile organic marker detection unit specifically may be adapted to determine an amount of the first volatile organic marker.

The at least one of the first volatile organic marker detection unit and the second volatile organic marker detection unit may be selected from the group consisting of: a gas sensor, exemplarily a metal oxide sensor; a carbon nanotube chip (CNT), specifically a functionalized carbon nanotube chip; a photometric sensor, exemplarily a light source detector based on absorption; a mass spectrometer, exemplarily an ion mobility spectrometer; an electrochemical sensor; a biochemical sensor with functionalized surfaces, wherein the functionalized surfaces may specifically comprise at least one of amino acids, peptides, proteins, DNA building blocks, other biomolecules or fragments thereof. Also other kinds of volatile organic marker detection units may be feasible. The gas sensor may specifically be a miniaturized sensor. Concerning the gas sensor, reference may be made to commercially available metal oxide sensors which are usable for monitoring indoor air and external air pollution.

The system may comprise at least one vapor distribution member configured for bringing a sample of exhaled breath or of another source of gaseous emanation from the subject in contact with the first volatile organic marker detection unit or the second volatile organic marker detection unit.

The term “evaluation device” as used herein is a broad term and is to be given its ordinary and customary meaning to a person of ordinary skill in the art and is not to be limited to a special or customized meaning. The term specifically may refer, without limitation, to an arbitrary component which is designed to actuate an arbitrary sensor and/or to record signals from the sensor and/or to derive at least one item of information of an analyte from the signals and/or to evaluate these signals in whole or part. Thus, the evaluation device may specifically be or may comprise an electronic component. The electronic component may be configured for one or more of performing a measurement with a sensor, performing a voltage measurement, performing a current measurement, recording sensor signals, storing measurement signals or measurement data, transmitting sensor signals or measurement data to another device. Thus, the electronic component specifically may comprise at least one of: a voltmeter, an amperemeter, a potentiostat, a voltage source, a current source, a signal receiver, a signal transmitter, an analog-digital converter, an electronic filter, an energy storage device, a data processing device, such as a microcontroller. Other embodiments of the electronic component are feasible. The electronics component may specifically comprise at least one circuit board having disposed thereon elements of the electronics component.

The first volatile organic marker detection unit and the second volatile organic marker detection unit may be connected or connectable to each other, specifically via wireless communication, preferably via wireless far-field communication, more preferably via radio frequency transmission. As used herein, the term “wireless far-field communication” generally refers to a wireless communication adapted to transmit data over long distances, such as distances of more than 10 cm. As an example, the wireless far-field communication may be an arbitrary long-range communication using electromagnetic waves in the radio frequency range, i.e., may be a radio communication. Thus, as an example, a wireless far-field communication may comprise at least one radio module, having at least one radio antenna, for transmitting data via radio transmission to another device.

Specifically, at least one of the first volatile organic marker detection unit and the second volatile organic marker detection unit may be configured for transferring data to at least one external device, specifically to at least one smartphone or to at least one smartwatch. As used herein, the term “external device” may be an arbitrary device adapted to receive data via wireless far-field communication from the first volatile organic marker detection unit and/or the second volatile organic marker detection unit. The at least one external device may be part of the system or may be independent from the system. As an example, the at least one external device may be a portable device having the capability of communicating via wireless far-field communication, such as a hand-held computer and/or a smartphone. Other examples are feasible.

The first volatile organic marker detection unit and the second volatile organic marker detection unit may be placeable at different positions of a subject's body. At least one of the first volatile organic marker detection unit and the second volatile organic marker detection unit may be arranged on one of a wristband, a patch placeable on a subject's skin such as via an adhesive, a pair of glasses, nose-clip, a nose-ring, a nose-piercing, a headband, a headset, a chest strap, a piece of clothing, a pillow, a device on a bedside table. Specifically, one of first volatile organic marker detection unit and the second volatile organic marker detection unit may be configured for establishing a skin contact with a subject's skin, preferably continuously and/or permanently, and another one of the first volatile organic marker detection unit and the second volatile organic marker detection unit may be configured for being arrangeable below a subject's nostril and/or below a subject's mouth, either continuously and/or permanently and/or discontinuously. At least one of the first volatile organic marker detection unit and the second volatile organic marker detection unit may be arranged on or integrated in a smartwatch.

The first volatile organic marker detection unit may be arranged on the wristband and may be configured for detecting the first volatile organic marker from the first source of the subject being exhaled breath, preferably discontinuously. Further, the system may comprise a vapor distribution member configured for bringing a sample of exhaled breath or of another source of gaseous emanation from the subject in contact with the first volatile organic marker detection unit. The second volatile organic marker detection unit may be arranged on the wristband, and the second volatile organic marker detection unit may be configured for detecting the first volatile organic marker from the second source of the subject being a subject's skin, preferably continuously. Specifically, both of the first volatile organic marker detection unit and the second volatile organic marker detection unit may be arranged on the wristband.

The sample of exhaled breath may be captured using a mouthpiece, a nasal cannula, a handheld breath analyzer or any other device suitable to capture at least a fraction of the exhaled breath.

The present disclosure further discloses and proposes a computer program including computer-executable instructions for performing the method according to the present disclosure in one or more of the embodiments enclosed herein when the program is executed on a computer or computer network. Specifically, the computer program may be stored on a computer-readable data carrier. Thus, specifically, one, more than one or even all of method steps a) to c) as indicated above may be performed by using a computer or a computer network, preferably by using a computer program.

The present disclosure further discloses and proposes a computer program product having program code means, in order to perform the method according to the present disclosure in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a computer or computer network, such as into a working memory or main memory of the computer or computer network, may execute the method according to one or more of the embodiments disclosed herein.

The present disclosure further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the present disclosure proposes and discloses a modulated data signal which contains instructions readable by a computer system or computer network, for performing the method according to one or more of the embodiments disclosed herein.

Preferably, referring to the computer-implemented aspects of the present disclosure, one or more of the method steps a) to c) or even all of the method steps a) to c) of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present disclosure further discloses:
a computer or computer network comprising at least one processor, wherein the processor is adapted to perform the method, specifically one or more the method steps a) to c), according to one of the embodiments described in this description,
a computer loadable data structure that is adapted to perform the method, specifically one or more the method steps a) to c), according to one of the embodiments described in this description while the data structure is being executed on a computer,
a computer program, wherein the computer program is adapted to perform the method, specifically one or more the method steps a) to c), according to one of the embodiments described in this description while the program is being executed on a computer,
a computer program comprising program means for performing the method, specifically one or more the method steps a) to c), according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method, specifically one or more the method steps a) to c), according to one of the embodiments described in this description after having been loaded into a main and/or working storage of a computer or of a computer network, and
a computer program product having program code means, wherein the program code means can be stored or are stored on a storage medium, for performing the method, specifically one or more the method steps a) to c), according to one of the embodiments described in this description, if the program code means are executed on a computer or on a computer network.

The proposed methods and devices show many advantages over known devices and methods.

The present disclosure relates to non-invasive analyte determination based on volatile organic compounds (VOCs). Measurements of the same volatile marker or the same combination of volatile markers originating from at least two different sources may be combined. Combining a measurement of a volatile organic marker from a first source such as breath with a measurement of the same marker originating from another source such as emanation such as sweat may significantly increase an accuracy of the analyte determination.

The combination of multiple sources for one or more volatile markers may significantly increase an accuracy of the non-invasive analyte determination based on the volatile marker(s). Further, it may allow for user-friendly measurement procedures such as combining continuous emanation monitoring via a noninvasive on skin sensor with punctual breath monitoring via a same or a different sensor device.

Several detectors, specifically sensors, may be used which may be placed at different positions of the subject's body. The detectors may be connected or connectable to each other such as via radio frequency transmission and may also be similarly connected or connectable to the external device such as to the smartphone. Specifically, one kind of VOC may be detected from all sources. Measurements may be performed continuously or as spot monitoring.

The smartwatch on the wrist may be configured for detecting continuously the VOCs from skin, specifically without displaying a glucose value. If the subject wants to make a glucose measurement such as a spot-monitoring the subject may move the arm with the smartwatch close to his/her mouth and may blow on it 2-3 times. The smartwatch may be configured for detecting this process and may be configured for evaluating the 2-3 exhaled breath cycles. The combined values of VOCS from breath and skin may be used to calculate a glucose value.

Glasses may be equipped with a sensor for VOCs originating from the skin on the frame. The sensor may have a skin contact. Further, additionally or alternatively, the glasses may have a similar sensor behind the ear. In addition, a sensor for VOCs originating from exhaled breath may be placed below the nostrils.

The nose-clip, the nose-ring and/or the nose-piercing may be equipped with a sensor for VOCs originating from exhaled breath and may be connected or connectable via radio to the smartwatch and/or smartring with a sensor for VOCs originating from the skin.

The smartwatch on the wrist or the smartring on the finger or patch on the skin in a different place for VOCs originating from the skin may be connected or connectable to a smartphone for VOCs originating from exhaled breath which may be used for spot-monitoring by blowing on it.

A headset with a sensor-arm for VOCs originating from exhaled breath such as similar to a microphone arm of common headsets and additional sensors on the cheek or behind the ear for VOCs originating from the skin may be realized. The sensor for VOCs originating from exhaled breath may be coupled or may be coupleable to additional sensors for VOCs originating from the skin on the wrist such as via the smartwatch or on the finger such as via the smartring.

Further, clothing may be equipped with detectors for VOCs originating from the skin. A detection system for VOCs originating from exhaled breath may be realized that is not a wearable but may be placeable near the subject to monitor the blood glucose values, e.g., a pillow and/or a device on a bedside table.

The system may comprise a screening alarm system which, specifically, does not give a continuous glucose value but only alerts the owner subject in case of values “out of range”, i.e., hyperglycemia or hypoglycemia. “Unique clusters”, i.e., dedicated groups of VOCs, may be used to specifically detect states of hyperglycemia or hypoglycemia. A blood glucose time profile may be recorded and may provide indications about actions. The subject may be informed about his/her glycemic condition, such as in the morning.

A continuous series of consecutive measurements may be used to support or to improve a prediction of glucose condition such as a trend in the near future such as within a few minutes. Machine learning such as including neuronal networks may be used as a mathematical model to improve an accuracy of glucose readings. Other mathematical evaluation models, using double-logarithmical functions may be applied.

A field calibration may be applied including a procedure to establish a personalized algorithm for a specific subject. An entire series of breath measurements may be used during an event with glucose variation (such as an oral glucose tolerance test OGTT. Only a few single breath measurements to classify and/or cluster patients to a specific algorithm, chosen from a number of readily pre-determined algorithm options may be applied. The clustering or grouping may be supported by other patient characteristics, not depending on their breath such as age, gender, BMI, weight and/or medication. Feedback to the patient about a goodness of fit to a specific algorithm may be provided, with options to improve it if needed (either do an OGTT or use a different CGM system). Failsafes, e.g., with regard to wrong results, using VOCs originating from exhaled breath and VOCs originating from the skin may be realized.

Summarizing and without excluding further possible embodiments, the following embodiments may be envisaged:

Embodiment 1: A method for determining a subject's analyte level, the method comprises the steps of:
a) non-invasively detecting a first amount of at least one first volatile organic marker originating from a first source of the subject;
b) non-invasively detecting a second amount of at least the first volatile organic marker originating from a second source of the subject, wherein the second source is different from the first source; and
c) determining the subject's analyte level based on the first amount and on the second amount.

Embodiment 2: The method according to the preceding embodiment, wherein the first source is exhaled breath and wherein the second source is another source of gaseous emanation which is different from exhaled breath or vice versa.

Embodiment 3: The method according to any one of the preceding embodiments, wherein the analyte is glucose, specifically blood glucose.

Embodiment 4: The method according to any one of the preceding embodiments, wherein the first volatile organic marker is selected from a group of markers for which the amount of the volatile organic marker is negatively correlated to the glucose level.

Embodiment 5: The method according to any one of the preceding embodiments, wherein the first volatile organic marker is one of indole (C₈H₇N), a partly saturated derivative of indole, fully saturated derivative of indole and a true fraction of indole.

Embodiment 6: The method according to any one of the preceding embodiments, wherein the first volatile organic marker is selected from the group consisting of: formaldehyde, methanol.

Embodiment 7: The method according to any one of the preceding embodiments, wherein the method further comprises:
d) non-invasively detecting a third amount of at least a second volatile organic marker originating from the first source of the subject; and
e) non-invasively detecting a fourth amount of the at least one second volatile organic marker originating from the second source of the subject;
   wherein in step c) the subject's analyte level is detected based on the first amount the second amount, the third amount and the fourth amount.

Embodiment 8: The method according to the preceding embodiment, wherein the first volatile organic marker is indole and the second volatile organic marker is formaldehyde or methanol.

Embodiment 9: The method according to any one of the two preceding embodiments, wherein the analyte is glucose, wherein the method further comprises detecting a state of hyperglycemia or hypoglycemia based on first amount, the second amount, the third amount and the fourth amount.

Embodiment 10: The method according to any one of the three preceding embodiments, wherein the method further comprises:
f) non-invasively detecting a fifth amount of at least a third volatile organic marker originating from the first source of the subject; and
g) non-invasively detecting a sixth amount of the at least one third volatile organic marker originating from the second source of the subject;
   wherein in step c) the subject's analyte level is detected based on the first amount the second amount, the third amount, the fourth amount, the fifth amount and the sixth amount.

Embodiment 11: The method according to the preceding embodiment, wherein the first volatile organic marker, the second volatile organic marker and the third volatile organic marker are formaldehyde, methanol and indole.

Embodiment 12: The method according to any one of the two preceding embodiments, wherein the analyte is glucose, wherein the method further comprises detecting a state of hyperglycemia or hypoglycemia based on first amount the second amount, the third amount, the fourth amount, the fifth amount and the sixth amount.

Embodiment 13: The method according to any one of the preceding embodiments, wherein in step a) the first amount of the at least one first volatile organic marker originating from the first source of the subject is detected continuously or discontinuously.

Embodiment 14: The method according to any one of the preceding embodiments, wherein in step b) the second amount of the at least one first volatile organic marker originating from the second source of the subject is detected continuously or discontinuously.

Embodiment 15: The method according to any one of the preceding embodiments, wherein in step a) the first amount of the at least one first volatile organic marker originating from the first source of the subject is detected continuously and wherein in step b) the second amount of the at least one first volatile organic marker originating from the second source of the subject is detected discontinuously.

Embodiment 16: The method according to any one of the preceding embodiments, wherein the method further comprises displaying the subject's analyte level to the subject.

Embodiment 17: The method according to any one of the preceding embodiments, wherein the method further comprises comparing the subject's analyte level to a predefined tolerance interval being defined by a first threshold value and a second threshold value being different from the first threshold value, wherein a warning signal is output in case the subject's analyte level is outside the predefined tolerance interval.

Embodiment 18: A system for determining a subject's analyte level, wherein the system is configured for executing the method according to any one of the preceding embodiments, wherein the system comprises:
a first volatile organic marker detection unit configured for non-invasively detecting the first amount of the at least one first volatile organic marker originating from the first source of the subject;
a second volatile organic marker detection unit configured for non-invasively detecting the second amount of the at least one first volatile organic marker originating from the second source of the subject; and
at least one evaluation device configured for determining the subject's analyte level based on the first amount and on the second amount.

Embodiment 19: The system according to the preceding embodiment, wherein the system comprises at least one vapor distribution member configured for bringing a sample of exhaled breath or of another source of gaseous emanation from the subject in contact with the first volatile organic marker detection unit or the second volatile organic marker detection unit.

Embodiment 20: The system according to any one of the two preceding embodiments, wherein the at least one of the first volatile organic marker detection unit and the second volatile organic marker detection unit are selected from the group consisting of: a gas sensor, exemplarily a metal oxide sensor; a carbon nanotube chip (CNT), specifically a functionalized carbon nanotube chip; a photometric sensor, exemplarily a light source detector based on absorption; a mass spectrometer, exemplarily an ion mobility spectrometer; an electrochemical sensor; a biochemical sensor with functionalized surfaces, wherein the functionalized surfaces may specifically comprise at least one of amino acids, peptides, proteins, DNA building blocks, other biomolecules or fragments thereof.

Embodiment 21: The system according to any one of the three preceding embodiments, wherein the first volatile organic marker detection unit and the second volatile organic marker detection unit are placeable at different positions of a subject's body.

Embodiment 22: The system according to any one of the four preceding embodiments, wherein the first volatile organic marker detection unit and the second volatile organic marker detection unit are connectable to each other, specifically via wireless communication, preferably via wireless far-field communication, more preferably via radio frequency transmission.

Embodiment 23: The system according to any one of the five preceding embodiments, wherein at least one of the first volatile organic marker detection unit and the second volatile organic marker detection unit is configured for transferring data to at least one external device, specifically to at least one smartphone.

Embodiment 24: The system according to any one of the six preceding embodiments, wherein at least one of the first volatile organic marker detection unit and the second volatile organic marker detection unit are arranged on one of a wristband, a patch placeable on a subject's skin such as via an adhesive, a pair of glasses, nose-clip, a nose-ring, a nose-piercing, a headband, a headset, a piece of clothing, a pillow, a device on a bedside table.

Embodiment 25: The system according to the preceding embodiment, wherein at least one of the first volatile organic marker detection unit and the second volatile organic marker detection unit are arranged on or integrated in a smartwatch.

Embodiment 26: The system according to any one of the two preceding embodiments, wherein the first volatile organic marker detection unit is arranged on the wristband, wherein the first volatile organic marker detection unit is configured for detecting the first volatile organic marker from the first source of the subject being exhaled breath, preferably discontinuously, wherein the system comprises a vapor distribution member configured for bringing a sample of exhaled breath or of another source of gaseous emanation from the subject in contact with the first volatile organic marker detection unit.

Embodiment 27: The system according to any one of the three preceding embodiments, wherein the second volatile organic marker detection unit is arranged on the wristband, wherein the second volatile organic marker detection unit is configured for detecting the first volatile organic marker from the second source of the subject being a subject's skin, preferably continuously.

Embodiment 28: The system according to any one of the four preceding embodiments, wherein both of the first volatile organic marker detection unit and the second volatile organic marker detection unit are arranged on the wristband.

Embodiment 29: The system according to any one of the eleven preceding embodiments, wherein one of first volatile organic marker detection unit and the second volatile organic marker detection unit are configured for establishing a skin contact with a subject's skin, preferably continuously and/or permanently, and wherein another one of the first volatile organic marker detection unit and the second volatile organic marker detection unit are configured for or being arrangeable below a subject's nostril, either continuously and/or permanently or discontinuously.

### BRIEF DESCRIPTION OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A, 1B, 1C and 1D show scatter plots illustrating a correlation between measured and predicted blood glucose values;

FIGS. 2A and 2B show a further scatter plot illustrating a correlation between measured and predicted blood glucose values (FIG. 2A) and a consensus error grid (FIG. 2B);

FIGS. 3A and 3B show a further scatter plot illustrating a correlation between measured and predicted blood glucose values (FIG. 3A) and a further consensus error grid (FIG. 3B);

FIGS. 4A and 4B show a summary of results of a scatter plot illustrating a correlation between measured and predicted blood glucose values (FIG. 4A) and further consensus error grid (FIG. 4B);

FIGS. 5A and 5B show a further scatter plot illustrating a correlation between measured and predicted blood glucose values (FIG. 5A) and a further consensus error grid (FIG. 5B);

FIGS. 6A and 6B show a further scatter plot illustrating a correlation between measured and predicted blood glucose values (FIG. 6A) and a further consensus error grid (FIG. 6B);

FIGS. 7A and 7B show two further scatter plots illustrating a correlation between measured and predicted blood glucose values; and

FIG. 8 shows a summary of results illustrated in FIGS. 2A, 3A, 4A, 5A, 6A, 7A and 7B.

Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

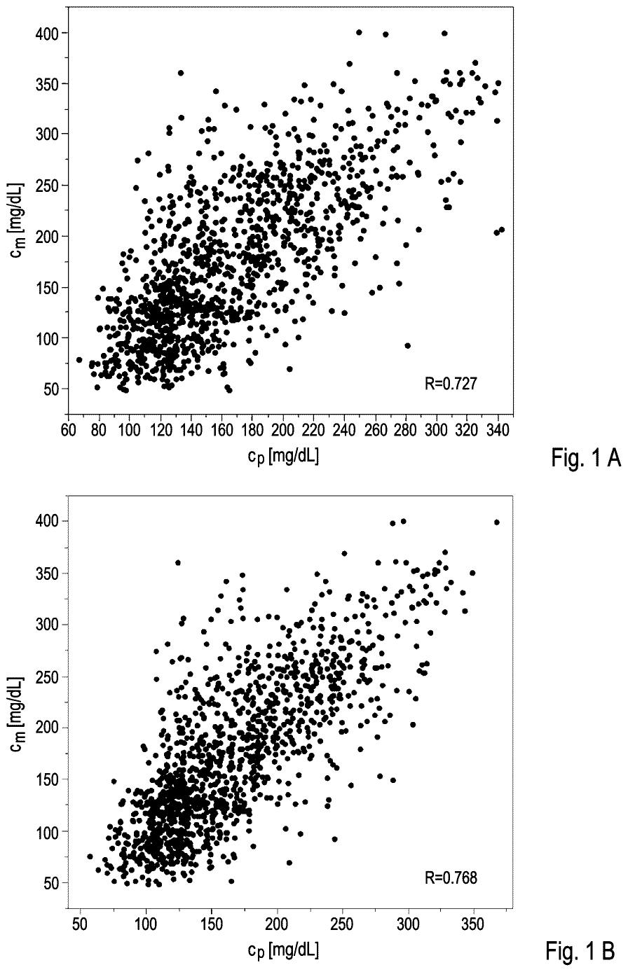

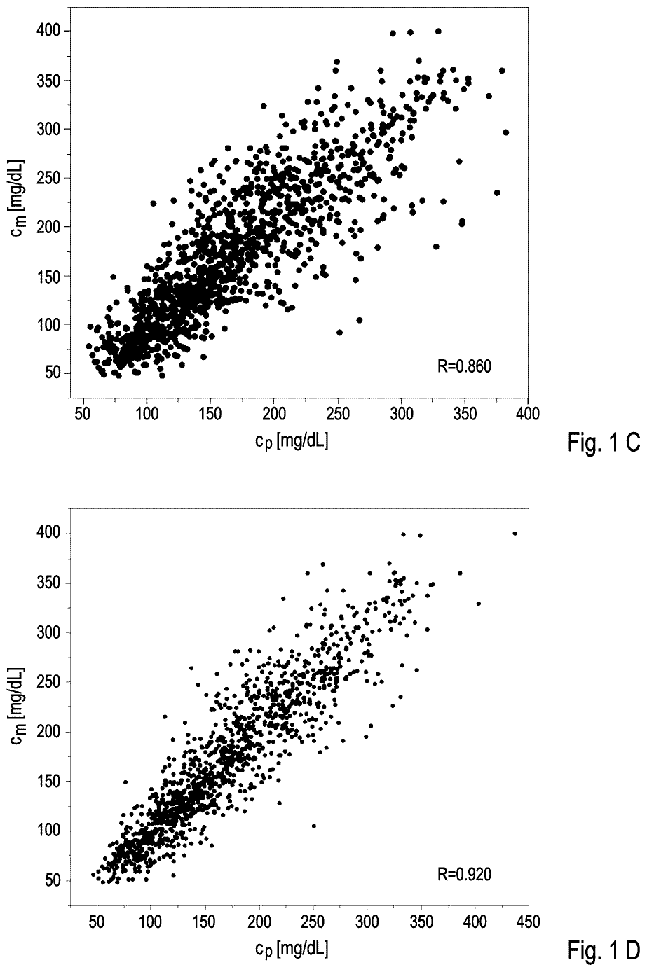

FIGS. 1A to 1D respectively show scatter plots illustrating a correlation between measured and predicted blood glucose values. In FIGS. 1A to 1D, on the y-axis measured blood glucose values Cm in mg/dl are respectively plotted. Further, in FIGS. 1A to 1D, on the x-axis predicted blood glucose values cp in mg/dL are respectively plotted. A Log-Log model was applied.

FIG. 1A shows a scatter plot wherein, for the mathematical model applied for determining the predicted blood glucose values, indole was applied as volatile organic marker originating from breath as source of a subject. A linear regression was conducted and the correlation coefficient R was 0.727. Thus, there may be a significant correlation of predicted and measured blood glucose values.

FIG. 1B shows a scatter plot wherein, for the mathematical model applied for determining the predicted blood glucose values, indole was applied as volatile organic marker originating from breath and skin as sources of a subject. A linear regression was conducted and R was 0.768. Thus, by detecting indole from two different kinds of sources, the correlation of predicted and measured blood glucose values increased.

FIG. 1C shows a scatter plot wherein, for the mathematical model applied for determining the predicted blood glucose values, indole, methanol and formaldehyde were applied as volatile organic markers originating from breath as source of a subject. A linear regression was conducted and R was 0.860. Thus, by detecting three different kinds of volatile organic markers, the correlation of predicted and measured blood glucose values increased.

FIG. 1D shows a scatter plot wherein, for the mathematical model applied for determining the predicted blood glucose values, indole, methanol and formaldehyde were applied as volatile organic markers originating from breath and skin as source of a subject. A linear regression was conducted and R was 0.920. Thus, by detecting three different kinds of volatile organic markers respectively from two different kinds of sources, the correlation of predicted and measured blood glucose values even increased further.

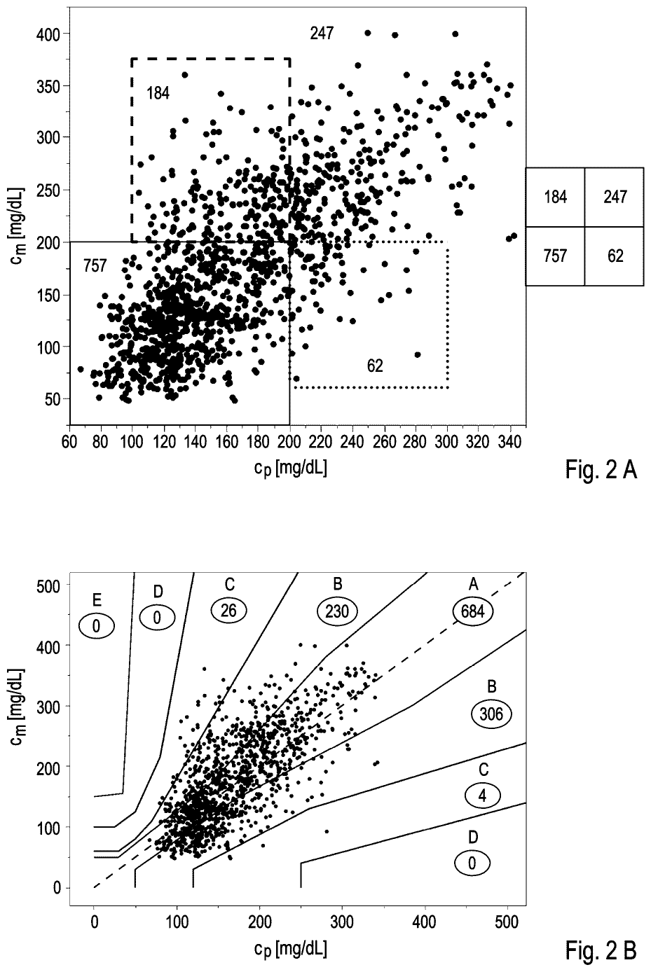

FIG. 2A shows a further scatter plot illustrating a correlation between measured and predicted blood glucose values and FIG. 2B shows a consensus error grid.

In FIG. 2A, on the y-axis measured blood glucose values cm in mg/dL are plotted. Further, on the x-axis predicted blood glucose values cp in mg/dl are plotted. For the mathematical model applied for determining the predicted blood glucose values, indole was the volatile organic marker originating from breath as source of a subject.

Alarms should be provided at high blood glucose values being larger than 200 mg/dL. Thus, in FIG. 2A, three boxes are illustrated. A lower left box is illustrated with solid lines. An upper left box is illustrated with dashed lines. A lower right box is illustrated with dotted lines.

The number of blood glucose counts in the study was 1250 originating from 50 patients, 25 observations each.

The lower left box comprises 757 counts corresponding to normal blood glucose values being smaller than 200 mg/dL. The upper let box comprises 184 counts corresponding to false negative results. Thus, mistakenly, no alarm is output. The lower right box comprises 62 counts corresponding to false positive results. Thus, mistakenly, an alarm is output. The residual 247 counts correspond to correct positive results.

Thus, when the subject is hyper, the model predicts correctly with 57% (247/(184+247)). Further, a hyper prediction of the model is correct with 80% (247/(247+62)).

FIG. 2B shows a consensus error grid wherein indole was the volatile organic marker originating from breath as source of a subject. The number of blood glucose counts in the study was 1250 originating from 50 patients, 25 observations each.

The consensus error grid, also known as Parkes error grid, was developed as a new tool for evaluating the accuracy of a blood glucose meter. In recent times, the consensus error grid has been used increasingly by blood glucose meter manufacturers in their clinical studies. The guidelines for ISO15197: 2013 specify the usage of the consensus error grid for evaluation of blood glucose monitoring systems. In zone A, there is commonly no effect on clinical action. In zone B, there is commonly little or no effect on clinical outcome. In zone C, it is commonly likely to affect clinical outcome. In zone D, there commonly could be a significant medical risk. In zone E, there commonly could be dangerous consequences.

As illustrated in FIG. 2B, zones A comprise 684 counts, zones B comprise 536 counts, zones C comprise 31 counts and zones D and E respectively comprise 0 counts.

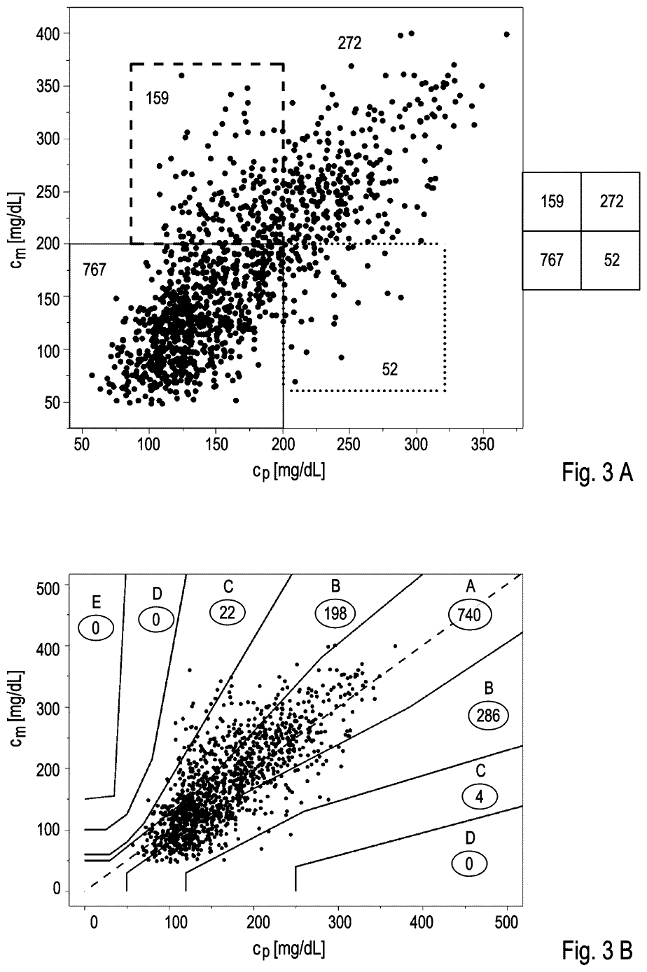

FIG. 3A shows a further scatter plot illustrating a correlation between measured and predicted blood glucose values and FIG. 3B shows a further consensus error grid.

In FIG. 3A, on the y-axis measured blood glucose values cm in mg/dl are plotted. Further, on the x-axis predicted blood glucose values cp in mg/dl are plotted. For the mathematical model applied for determining the predicted blood glucose values, indole was the volatile organic marker originating from breath and skin as sources of a subject.

Alarms should be provided at high blood glucose values being larger than 200 mg/dL. Thus, in FIG. 2A, three boxes are illustrated. A lower left box is illustrated with solid lines. An upper left box is illustrated with dashed lines. A lower right box is illustrated with dotted lines. The number of blood glucose counts in the study was 1250 originating from 50 patients, 25 observations each.

The lower left box comprises 767 counts corresponding to normal blood glucose values being smaller than 200 mg/dL. The upper left box comprises 154 counts corresponding to false negative results. Thus, mistakenly, no alarm is output. The lower right box comprises 52 counts corresponding to false positive results. Thus, mistakenly, an alarm is output. The residual 272 counts correspond to correct positive results.

Thus, when the subject is hyper, the model predicts correctly with 63% (272/(159+272)). Further, a hyper prediction of the model is correct with 84% (272/(272+52)).

FIG. 3B shows a consensus error grid wherein indole was the volatile organic marker originating from breath and skin as sources of a subject. The number of blood glucose counts in the study was 1250 originating from 50 patients, 25 observations each.

As illustrated in FIG. 3B, zones A comprise 740 counts, zones B comprise 484 counts, zones C comprise 26 counts and zones D and E respectively comprise 0 counts.

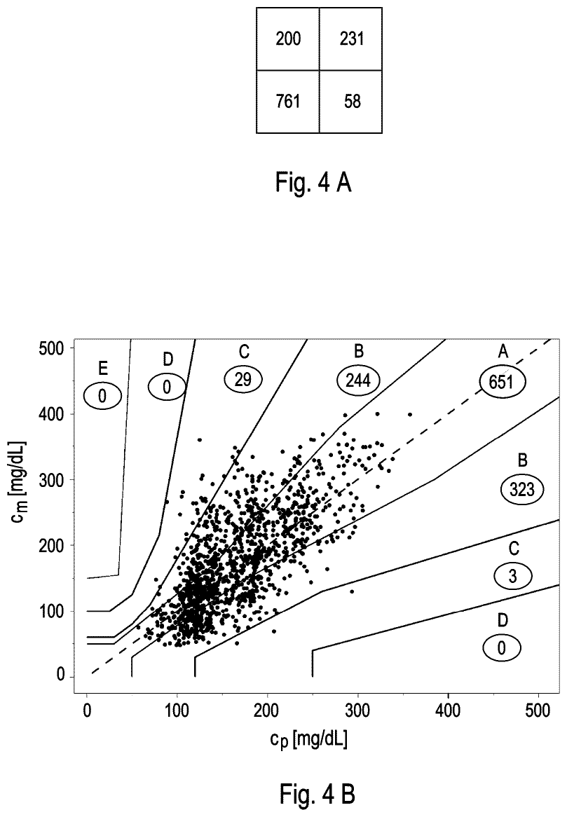

FIG. 4A shows a summary of results of a further scatter plot illustrating a correlation between measured and predicted blood glucose values and FIG. 4B shows a further consensus error grid.

For the results illustrated in FIGS. 4A and 4B, for the mathematical model applied for determining the predicted blood glucose values, indole was the volatile organic marker originating from skin as source of a subject. The number of blood glucose counts in the study was 1250 originating from 50 patients, 25 observations each.

Alarms should be provided at high blood glucose values being larger than 200 mg/dL. Thus, in FIG. 4A, four boxes are illustrated. The lower left box comprises 761 counts corresponding to normal blood glucose values being smaller than 200 mg/dL. The upper left box comprises 200 counts corresponding to false negative results. Thus, mistakenly, no alarm is output. The lower right box comprises 58 counts corresponding to false positive results. Thus, mistakenly, an alarm is output. The residual 231 counts (upper right box) correspond to correct positive results.

Thus, when the subject is hyper, the model predicts correctly with 54% (231/(231+200)). Further, a hyper prediction of the model is correct with 80% (231/(231+58)).

FIG. 4B shows a consensus error grid. As illustrated in FIG. 4B, zones A comprise 651 counts, zones B comprise 567 counts, zones C comprise 32 counts and zones D and E respectively comprise 0 counts.

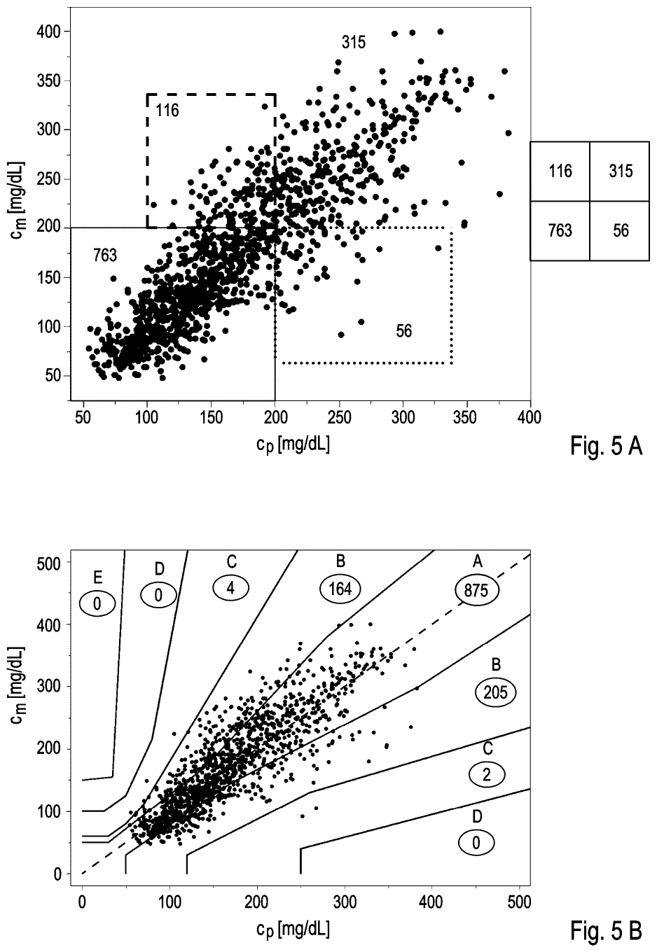

FIG. 5A shows a further scatter plot illustrating a correlation between measured and predicted blood glucose values and FIG. 5B shows a further consensus error grid.

In FIG. 5A, on the y-axis measured blood glucose values cm in mg/dl are plotted. Further, on the x-axis predicted blood glucose values cp in mg/dl are plotted. For the mathematical model applied for determining the predicted blood glucose values, indole, formaldehyde and methanole were the volatile organic markers originating from breath sources of a subject.

Alarms should be provided at high blood glucose values being larger than 200 mg/dL. Thus, in FIG. 5A, three boxes are illustrated. A lower left box is illustrated with solid lines. An upper left box is illustrated with dashed lines. A lower right box is illustrated with dotted lines. The number of blood glucose counts in the study was 1250 originating from 50 patients, 25 observations each.

The lower left box comprises 763 counts corresponding to normal blood glucose values being smaller than 200 mg/dL. The upper left box comprises 116 counts corresponding to false negative results. Thus, mistakenly, no alarm is output. The lower right box comprises 56 counts corresponding to false positive results. Thus, mistakenly, an alarm is output. The residual 315 counts correspond to correct positive results.

Thus, when the subject is hyper, the model predicts it with 73% (315/(315+56)) Further, a hyper prediction of the model is correct with 85% (315/(315+116)).

FIG. 5B shows a consensus error grid wherein indole, formaldehyde and methanole were the volatile organic markers originating from breath as source of a subject. The number of blood glucose counts in the study was 1250 originating from 50 patients, 25 observations each.

As illustrated in FIG. 5B, zones A comprise 875 counts, zones B comprise 369 counts, zones C comprise 6 counts and zones D and E respectively comprise 0 counts.

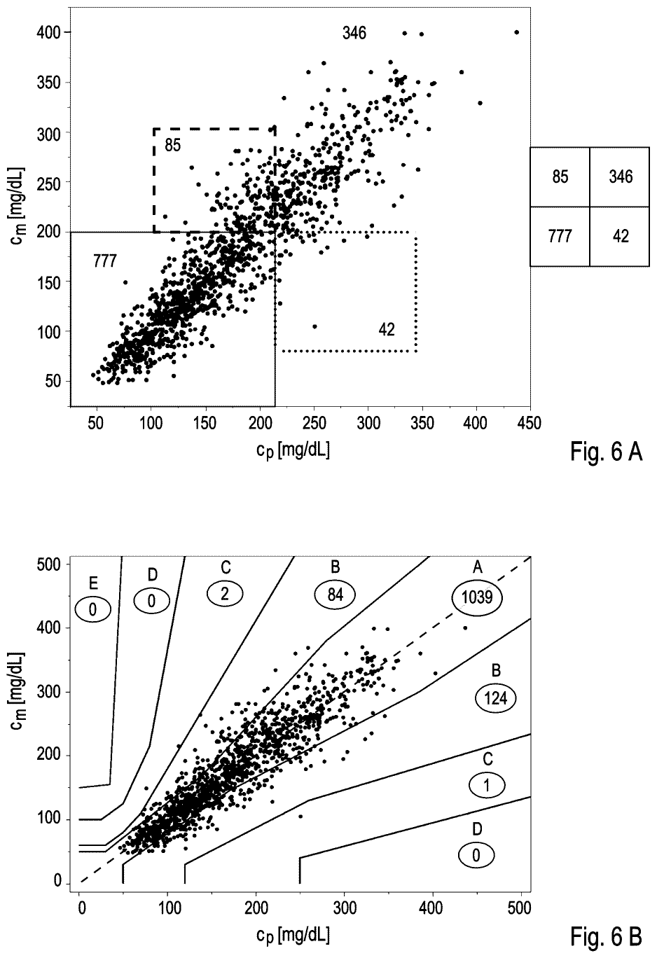

FIG. 6A shows a further scatter plot illustrating a correlation between measured and predicted blood glucose values and FIG. 6B shows a further consensus error grid.

In FIG. 6A, on the y-axis measured blood glucose values cm in mg/dl are plotted. Further, on the x-axis predicted blood glucose values cp in mg/dl are plotted. For the mathematical model applied for determining the predicted blood glucose values, indole, formaldehyde and methanole were the volatile organic markers originating from breath and skin as sources of a subject.

Alarms should be provided at high blood glucose values being larger than 200 mg/dL. Thus, in FIG. 6A, three boxes are illustrated. A lower left box is illustrated with solid lines. An upper left box is illustrated with dashed lines. A lower right box is illustrated with dotted lines. The number of blood glucose counts in the study was 1250 originating from 50 patients, 25 observations each.

The lower left box comprises 777 counts corresponding to normal blood glucose values being smaller than 200 mg/dL. The upper left box comprises 85 counts corresponding to false negative results. Thus, mistakenly, no alarm is output. The lower right box comprises 42 counts corresponding to false positive results. Thus, mistakenly, an alarm is output. The residual 346 counts correspond to correct positive results.

Thus, when the subject is hyper, the model predicts it with 80% (346/(346+86)) Further, a hyper prediction of the model is correct with 89% (346/(346+42)).

FIG. 6B shows a consensus error grid wherein indole, formaldehyde and methanole were the volatile organic markers originating from breath and skin as sources of a subject. The number of blood glucose counts in the study was 1250 originating from 50 patients, 25 observations each.

As illustrated in FIG. 6B, zones A comprise 1039 counts, zones B comprise 208 counts, zones C comprise 3 counts and zones D and E respectively comprise 0 counts.

In the following, Table 1 gives a summary on the results of the consensus error grids illustrated in FIGS. 2B, 3B, 4B, 5B and 6B.

TABLE 1
summary on the results of the consensus error grids illustrated
in FIGS. 2B, 3B, 4B, 5B and 6B, number of counts in the respective
zone are given as total numbers and in percent:

| | zone A | zone B | zone C | zones C/D |
|---|---|---|---|---|
| indole originating from | 684 (55%) | 536 (43%) | 30 (2%) | 0 (0%) |
| breath | | | | |
| indole originating from | 740 (59%) | 484 (39%) | 26 (2%) | 0 (0%) |
| breath and skin | | | | |
| indole originating from | 651 (52%) | 567 (45%) | 32 (3%) | 0 (0%) |
| skin | | | | |
| indole, formaldehyde and | 875 (70%) | 369 (30%) | 6 (0%) | 0 (0%) |
| methanol originating | | | | |
| from breath | | | | |
| indole, formaldehyde and | 1039 (83%) | 208 (17%) | 3 (0%) | 0 (0%) |
| methanol originating | | | | |
| from breath and skin | | | | |

This summary demonstrates that, the more different kinds of volatile organic markers and the more kinds of sources are considered, the percentage of correctly predicted hyperglycemic events can be improved.

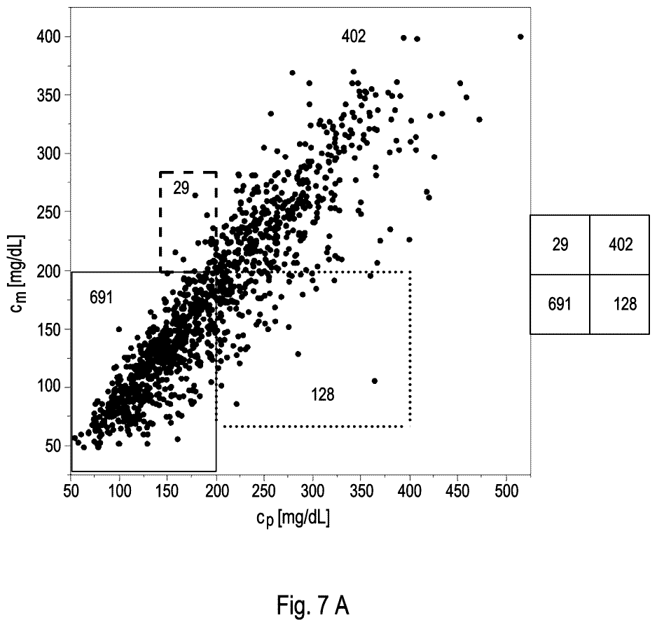

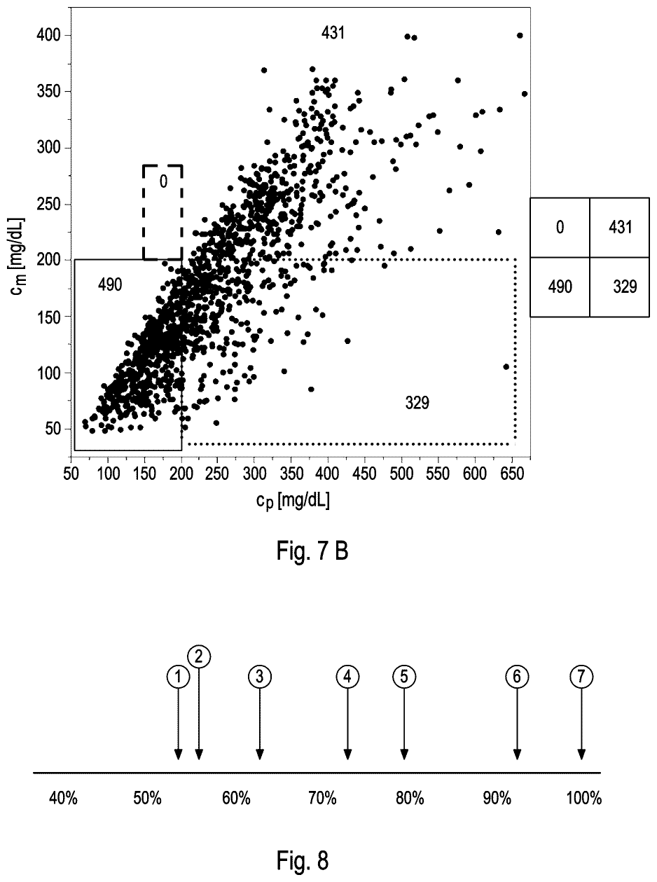

FIGS. 7A and 7B respectively show further scatter plots illustrating a correlation between measured and predicted blood glucose values.

In FIGS. 7A and 7B, on the y-axis measured blood glucose values cm in mg/dl are plotted. Further, on the x-axis predicted blood glucose values cp in mg/dL are plotted. For the mathematical model applied for determining the predicted blood glucose values, indole, formaldehyde and methanole were the volatile organic markers originating from breath and skin as sources of a subject. For FIG. 7A an upper 50% confidence interval was considered. For FIG. 7B an upper 90% confidence interval was considered.

Alarms should be provided at high blood glucose values being larger than 200 mg/dL. Thus, in FIGS. 7A and 7B, three boxes are illustrated. A lower left box is illustrated with solid lines. An upper left box is illustrated with dashed lines. A lower right box is illustrated with dotted lines. The number of blood glucose counts in the study was 1250 originating from 50 patients, 25 observations each.

In FIG. 7A, the lower left box comprises 691 counts corresponding to normal blood glucose values being smaller than 200 mg/dL. The upper left box comprises 29 counts corresponding to false negative results. Thus, mistakenly, no alarm is output. The lower right box comprises 128 counts corresponding to false positive results. Thus, mistakenly, an alarm is output. The residual 402 counts correspond to correct positive results. Thus, when the subject is hyper, the model predicts it with 93% (402/(402+29)). Further, a hyper prediction of the model is correct with 76% (402/(402+129)).

In FIG. 7B, the lower left box comprises 490 counts corresponding to normal blood glucose values being smaller than 200 mg/dL. The upper left box comprises 0 counts corresponding to false negative results. Thus, mistakenly, no alarm is output. The lower right box comprises 329 counts corresponding to false positive results. Thus, mistakenly, an alarm is output. The residual 431 counts correspond to correct positive results. Thus, when the subject is hyper, the model predicts it with 100% (431/(431+0)). Further, a hyper prediction of the model is correct with 57% (431/(431+329)).

FIG. 8 shows a summary of results illustrated in FIGS. 2A, 3A, 4A, 5A, 6A, 7A and 7B. Specifically, FIG. 8 shows a percent of hyperglycemic events wherein the blood glucose concentration is larger than 200 mg/dL correctly predicted in a study with 50 patients.

The arrow marked with “1” represents the results illustrated in FIG. 4A. Thus, when indole is the volatile organic marker originating from skin as source of a subject for the mathematical model applied for determining the predicted blood glucose values, the percent of hyperglycemic events correctly predicted is 54%.

The arrow marked with “2” represents the results illustrated in FIG. 2A. Thus, when indole is the volatile organic marker originating breath as source of a subject for the mathematical model applied for determining the predicted blood glucose values, the percent of hyperglycemic events correctly predicted is 57%.

The arrow marked with “3” represents the results illustrated in FIG. 3A. Thus, when indole is the volatile organic marker originating from skin and breath as sources of a subject for the mathematical model applied for determining the predicted blood glucose values, the percent of hyperglycemic events correctly predicted is 63%.

The arrow marked with “4” represents the results illustrated in FIG. 5A. Thus, when indole, formaldehyde and methanol are the volatile organic marker originating from breath as source of a subject for the mathematical model applied for determining the predicted blood glucose values, the percent of hyperglycemic events correctly predicted is 73%.

The arrow marked with “5” represents the results illustrated in FIG. 6A. Thus, when indole, formaldehyde and methanol are the volatile organic marker originating from breath and skin as sources of a subject for the mathematical model applied for determining the predicted blood glucose values, the percent of hyperglycemic events correctly predicted is 80%.

The arrow marked with “6” represents the results illustrated in FIG. 7A. Thus, when indole, formaldehyde and methanol are the volatile organic marker originating from breath and skin as sources of a subject for the mathematical model applied for determining the predicted blood glucose values, and when an upper 50% confidence interval is applied the percent of hyperglycemic events correctly predicted is 93%.

The arrow marked with “7” represents the results illustrated in FIG. 7B. Thus, when indole, formaldehyde and methanol are the volatile organic marker originating from breath and skin as sources of a subject for the mathematical model applied for determining the predicted blood glucose values, and when an upper 90% confidence interval is applied the percent of hyperglycemic events correctly predicted is 100%.

This summary also demonstrates that, the more different kinds of volatile organic markers and the more kinds of sources are considered, the percentage of correctly predicted hyperglycemic events can be improved. Further improvements may be achieved under consideration of a confidence interval.

While this invention has been described as having an exemplary design, the present invention may be further modified within the spirit and scope of this disclosure. This application is therefore intended to cover any variations, uses, or adaptations of the invention using its general principles.

What is claimed is:

1. A method for determining a subject's analyte level, the method comprises the steps of:
a) non-invasively detecting a first amount of at least one first volatile organic marker originating from a first source of the subject;
b) non-invasively detecting a second amount of at least the first volatile organic marker originating from a second source of the subject, wherein the second source is different from the first source; and
c) determining the subject's analyte level based on the first amount and on the second amount.

2. The method according to claim 1, wherein the first source is exhaled breath and wherein the second source is another source of gaseous emanation which is different from exhaled breath.

3. The method according to claim 1, wherein the analyte is glucose.

4. The method according to claim 1, wherein the first volatile organic marker is selected from a group of markers for which the amount of the volatile organic marker is negatively correlated to the glucose level.

5. The method according to claim 1, wherein the first volatile organic marker is one of indole (C₈H₇N), a partly saturated derivative of indole, fully saturated derivative of indole and a true fraction of indole.

6. The method according to claim 1, wherein the first volatile organic marker is selected from the group consisting of: formaldehyde, methanol.

7. The method according to claim 1, wherein the method further comprises:
d) non-invasively detecting a third amount of at least a second volatile organic marker originating from the first source of the subject; and
e) non-invasively detecting a fourth amount of the at least one second volatile organic marker originating from the second source of the subject;
wherein in step c) the subject's analyte level is detected based on the first amount, the second amount, the third amount, and the fourth amount.

8. The method according to claim 1, wherein the method further comprises:
d) non-invasively detecting a third amount of at least a second volatile organic marker originating from the first source of the subject; and
e) non-invasively detecting a fourth amount of the at least one second volatile organic marker originating from the second source of the subject;
wherein in step c) the subject's analyte level is detected based on the first amount, the second amount, the third amount, and the fourth amount, and wherein the first volatile organic marker is indole and the second volatile organic marker is formaldehyde or methanol.

9. The method according to claim 7, wherein the method further comprises:
f) non-invasively detecting a fifth amount of at least a third volatile organic marker originating from the first source of the subject; and
g) non-invasively detecting a sixth amount of the at least one third volatile organic marker originating from the second source of the subject;
wherein in step c) the subject's analyte level is detected based on the first amount, the second amount, the third amount, the fourth amount, the fifth amount, and the sixth amount.

10. The method according to claim 8 wherein the method further comprises:
f) non-invasively detecting a fifth amount of at least a third volatile organic marker originating from the first source of the subject; and
g) non-invasively detecting a sixth amount of the at least one third volatile organic marker originating from the second source of the subject;
wherein in step c) the subject's analyte level is detected based on the first amount, the second amount, the third amount, the fourth amount, the fifth amount, and the sixth amount, and wherein the first volatile organic marker, the second volatile organic marker and the third volatile organic marker are formaldehyde, methanol and indole.

11. The method according to claim 1, wherein in step a) the first amount of the at least one first volatile organic marker originating from the first source of the subject is detected continuously and wherein in step b) the second amount of the at least one first volatile organic marker originating from the second source of the subject is detected discontinuously.

12. A system for determining a subject's analyte level, wherein the system is configured for executing the method according to claim 1, wherein the system comprises:
a first volatile organic marker detection unit configured for non-invasively detecting the first amount of the at least one first volatile organic marker originating from the first source of the subject;
a second volatile organic marker detection unit configured for non-invasively detecting the second amount of the at least one first volatile organic marker originating from the second source of the subject; and
at least one evaluation device configured for determining the subject's analyte level based on the first amount and on the second amount.

13. The system according to claim 12, wherein the first volatile organic marker detection unit and the second volatile organic marker detection unit are placeable at different positions of a subject's body.

14. The system according to claim 12, wherein the first volatile organic marker detection unit and the second volatile organic marker detection unit are connectable to each other, and wherein at least one of the first volatile organic marker detection unit and the second volatile organic marker detection unit is configured for transferring data to at least one external device.

15. The system according to claim 14, wherein the first volatile organic marker detection unit and the second volatile organic marker detection unit are connectable to each other via wireless communication.

16. The system according to claim 14, wherein the first volatile organic marker detection unit and the second volatile organic marker detection unit are connectable to each other via wireless far-field communication.

17. The system according to claim 12, wherein one of first volatile organic marker detection unit and the second volatile organic marker detection unit are configured for establishing a skin contact with a subject's skin and wherein another one of the first volatile organic marker detection unit and the second volatile organic marker detection unit are configured for or being arrangeable below a subject's nostril or below a subject's mouth.
